(19) United States
(12) Patent Application Publication
(10) Pub. No.: US 2025/0352218 A1
(43) Pub. Date: Nov. 20, 2025

(54) HEMOSTATIC CLIP FOR ENDOSCOPE

(71) Applicant: Micro-Tech (Nanjing) Co., Ltd., Nanjing (CN)

(72) Inventors: Zhi Tang, Nanjing (CN); Chao Li, Nanjing (CN)

(21) Appl. No.: 19/207,578

(22) Filed: May 14, 2025

(30) Foreign Application Priority Data
May 17, 2024 (CN) 2024210873056

(51) Int. Cl. A61B 17/122; A61B 17/12; A61B 17/128

(52) U.S. Cl. CPC A61B 17/122; A61B 17/1285; A61B 2017/12004

(57) ABSTRACT

Hemostatic clip for endoscope is provided, including a clip holder and a clamping assembly, the clip holder has a passage provided to extend therethrough, a guiding slot communicating with the passage and an avoidance slot provided circumferentially along the clip holder and spaced apart from the guiding slot, the clip holder includes a first limiting portion, at least part of the first limiting portion is located in the guiding slot, and at least part of the first limiting portion is located between the guiding slot and the avoidance slot; and at least part of the clamping assembly is located in the passage, and the clamping assembly includes a movable pin and two clips connected to each other, with the movable pin threaded through the guiding slot; the clamping assembly can move relative to the clip holder along the passage, enabling the movable pin to move along the guiding slot.

### CROSS-REFERENCE TO RELATED APPLICATION

The present disclosure claims priority to Chinese patent application No. 2024210873056 filed with the China National Intellectual Property Administration on May 17, 2024, entitled “HEMOSTATIC CLIP FOR ENDOSCOPE”, the entire contents of which are incorporated herein by reference.

### TECHNICAL FIELD

The present disclosure belongs to the technical field of medical instruments, and specifically relates to a hemostatic clip for an endoscope.

### BACKGROUND ART

Hemostasis with hemostatic clips is categorized as mechanical hemostasis, that is, a tissue surrounding a wound and bleeding blood vessels are clamped and secured through a closing force between clamping pieces of the hemostatic clip, thereby achieving a hemostatic effect. In practical use, the hemostatic clip is delivered into a lesion site through a channel of an endoscope, the hemostatic clip is utilized to ligate the wound, and then the hemostatic clip is released so as to achieve a purpose of sustained hemostasis.

However, when hemostasis is performed on a large wound, due to a large wound area or rigid scar tissue, tension of wound tissue is high so that it is difficult for the hemostatic clip to clamp, and even if the wound tissue is clamped by the hemostatic clip, the tissue tension will cause insecure clamping of the hemostatic clip, further causing loosening and dropping off, resulting in delayed bleeding.

### SUMMARY

Objective of invention: The present disclosure provides a hemostatic clip for an endoscope, for solving the technical problem of loosening and dropping off caused by insecure clamping of the hemostatic clip in hemostasis of a large wound.

Technical solution: the present disclosure provides a hemostatic clip for an endoscope, including:
a clip holder, where the clip holder includes a passage that runs therethrough, a guiding slot communicating with the passage, and an avoidance slot provided circumferentially along the clip holder and spaced apart from the guiding slot, where the clip holder includes a first limiting portion, at least part of the first limiting portion is located in the guiding slot, and at least part of the first limiting portion is located between the guiding slot and the avoidance slot; and
a clamping assembly, where at least part of the clamping assembly is located in the passage, and the clamping assembly includes a movable pin and at least two clips, where the movable pin and the at least two clips are connected to each other, and the movable pin extends through the guiding slot; the clamping assembly can move relative to the clip holder along the passage, thereby enabling the movable pin to move along the guiding slot.

Herein, the movable pin can move along the guiding slot to a locking position, the first limiting portion is configured to deform towards the interior of the avoidance slot when subjected to compression of the movable pin, and the first limiting portion is configured to limit the movable pin to the locking position, so that the at least two clips are closed to each other and locked relative to a position of the clip holder.

In some embodiments, the first limiting portion is configured to generate elastic deformation when subjected to compression of the movable pin, so as to make the movable pin movable along the guiding slot and detach from the first limiting portion.

In some embodiments, the clip holder includes a plurality of first limiting portions, at least part of the guiding slot is provided between two first limiting portions opposite to each other; and

the avoidance slots are provided on two sides of the guiding slot, and each of the first limiting portions is corresponding to one avoidance slot.

In some embodiments, the locking position is located at a proximal end of the guiding slot.

In some embodiments, the hemostatic clip for an endoscope further includes:
a control assembly, where at least part of the control assembly is located in the passage, and the movable pin is connected to the control assembly; and the control assembly can move relative to the clip holder along the passage, so as to drive the clamping assembly to move.

In some embodiments, the movable pin includes a connecting portion and two second limiting portions, and two ends of the connecting portion are connected to the two second limiting portions, respectively; and
the clips and the control assembly are all connected to the connecting portion, and the second limiting portions are configured to limit the clips and the control assembly on the connecting portion in an extending direction of the movable pin.

In some embodiments, the control assembly includes a pulling wire (cable) and a clevis (buckle), the movable pin is connected to the clevis, the clevis has a first fitting hole, and the pulling wire is embedded in the first fitting hole so as to be connected to the clevis; and
when the movable pin is located in the locking position and the pulling wire is subjected to a preset tensile force, the pulling wire can be detached from the first fitting hole, so as to make the pulling wire disconnect from the clevis.

In some embodiments, the control assembly has a second fitting hole and a through slot communicating with the second fitting hole, the through slot extends from the fitting hole towards a distal end of the control assembly, and the movable pin extends through the second fitting hole; and
when the movable pin is located in the locking position and the control assembly is subjected to a preset tensile force, the control assembly can move relative to the movable pin along the through slot, so as to make the movable pin separate from the control assembly via the through slot.

In some embodiments, a width of the movable pin is greater than that of the through slot, and when the movable pin is located in the through slot, the movable pin compresses the control assembly to cause the control assembly to generate deformation, thereby enabling the movable pin to be detached from the through slot and separated from the control assembly.

In some embodiments, the control assembly has an elongate slot communicating with a side of the second fitting hole away from the through slot, and the elongate slot extends from the fitting hole towards a proximal end of the control assembly.

In some embodiments, the control assembly has a stopper hole communicating with the elongate slot, and the stopper hole communicates with a side of the elongate slot away from the second fitting hole.

In some embodiments, the hemostatic clip for an endoscope includes a catheter and a locking member, the control assembly extends through lumen of the catheter, the catheter has a first connection hole, the clip holder has a second connection hole, and the locking member extends through the first connection hole and the second connection hole so as to connect the catheter and the clip holder; and

when the movable pin is located in the locking position and the control assembly is subjected to a preset tensile force, the control assembly can abut against and push the locking member, so as to make the locking member detach from the first connection hole and the second connection hole, and make the catheter separate from the clip holder.

Beneficial effects: compared with the prior art, the hemostatic clip for an endoscope provided by the embodiments of the present disclosure includes a clip holder and a clamping assembly, where the clip holder has a passage provided to extend therethrough, a guiding slot communicating with the passage and an avoidance slot provided circumferentially along the clip holder and spaced apart from the guiding slot, the clip holder includes a first limiting portion, at least part of the first limiting portion is located in the guiding slot, and at least part of the first limiting portion is located between the guiding slot and the avoidance slot; and at least part of the clamping assembly is located in the passage, and the clamping assembly includes a movable pin and at least two clips connected to each other, with the movable pin extending through the guiding slot; the clamping assembly can move relative to the clip holder along the passage, thereby enabling the movable pin to move along the guiding slot. Herein, the movable pin can move along the guiding slot to a locking position, the first limiting portion is configured to deform towards the interior of the avoidance slot when subjected to compression of the movable pin, and the first limiting portion is configured to limit the movable pin to the locking position, thereby enabling the at least two clips to be closed to each other and locked relative to a position of the clip holder. In the present disclosure, by providing the first limiting portion and providing at least part of the first limiting portion in the guiding slot, the movable pin can be limited in the locking position, such that the clamp pieces, upon release, can maintain a persistently closed state relative to each other, and can avoid occurrence of delayed dropping off during hemostasis of large wounds with the hemostatic clip.

### BRIEF DESCRIPTION OF DRAWINGS

Below, technical solutions of the present disclosure and other beneficial effects will become obvious through detailed description of embodiments of the present disclosure in conjunction with drawings.

FIG. 1 is a structural schematic diagram of a hemostatic clip for an endoscope provided by embodiments of the present disclosure;

FIG. 2 is a cross-sectional view of the hemostatic clip for an endoscope provided by embodiments of the present disclosure in one direction;

FIG. 3 is a cross-sectional view of the hemostatic clip for an endoscope provided by embodiments of the present disclosure in another direction;

FIG. 4 is an exploded schematic view of the hemostatic clip for an endoscope provided by embodiments of the present disclosure;

FIG. 5 is a structural schematic diagram of a clip holder in the hemostatic clip for an endoscope provided by embodiments of the present disclosure;

FIG. 6 is a structural schematic diagram of a catheter in the hemostatic clip for an endoscope provided by embodiments of the present disclosure;

FIG. 7 is a structural schematic diagram of a control assembly in the hemostatic clip for an endoscope provided by embodiments of the present disclosure;

FIG. 8 is a structural schematic diagram of a movable pin in the hemostatic clip for an endoscope provided by embodiments of the present disclosure;

FIG. 9 is a structural schematic diagram of a locking member in the hemostatic clip for an endoscope provided by embodiments of the present disclosure;

FIG. 10 is a structural schematic diagram of a clevis in the hemostatic clip for an endoscope provided by embodiments of the present disclosure;

FIG. 11 is a structural schematic diagram of the hemostatic clip for an endoscope provided by embodiments of the present disclosure, after deformation of the clevis;

FIG. 12 is a structural schematic diagram of the hemostatic clip for an endoscope provided by embodiments of the present disclosure, with the movable pin being located in a locking position;

FIG. 13 is a cross-sectional view of the hemostatic clip for an endoscope provided by embodiments of the present disclosure, with the movable pin being located in the locking position; and

FIG. 14 is a cross-sectional view of the hemostatic clip for an endoscope provided by embodiments of the present disclosure, with the movable pin being separated from the control assembly.

Reference signs, 1—clip holder, 11—passage, 12—guiding slot, 13—first limiting portion, 14—avoidance slot, 15—second connection hole, 2—clamping assembly, 21—movable pin, 211—connecting portion, 212—second limiting portion, 22—clip piece, 3—control assembly, 31—pulling wire, 32—clevis, 321—first fitting hole, 33—second fitting hole, 34—through slot, 35—elongate slot, 36—stopper hole, 4—catheter, 41—first connection hole, 5—locking member, 51—jaw, 52—base, 53—through hole.

### DETAILED DESCRIPTION OF EMBODIMENTS

Technical solutions in embodiments of the present disclosure will be described clearly and completely below in conjunction with drawings in the embodiments of the present disclosure. Apparently, some but not all embodiments of the present disclosure are described. Based on the embodiments in the present disclosure, all of other embodiments obtained by those skilled in the art without using any inventive efforts shall fall within the scope of protection of the present disclosure.

In the description of the present disclosure, it should be indicated that, unless otherwise specifically regulated and defined, the terms “link”, and “connect” should be understood in a broad sense, for example, a connection may be a fixed connection, a detachable connection, or an integrated connection; it may be a mechanical connection or an electrical connection or mutual communication; it may be direct joining or indirect joining through an intermediary, and it also may be internal communication or interaction relationship between two elements. For those ordinarily skilled in the art, specific meanings of the above-mentioned terms in the present disclosure could be understood according to specific circumstances. In the description of the present disclosure, “a plurality of” means two or more, unless otherwise clearly and specifically specified. Besides, the terms “first” and “second” are merely used for descriptive purpose, but should not be construed as indicating or implying importance in the relativity or implicitly indicating the number of a related technical feature. Thus, defining a feature with “first” or “second” may explicitly or implicitly mean that one or more such features are included.

In the description of the present disclosure, it should be understood that the terms “proximal end” and “distal end” are used by an operator (surgeon) as a reference object, where the “proximal end” is an end of a surgical tool closer to the surgeon, and the “distal end” is an end farther from the surgeon than the “proximal end”, i.e., an end closer to a patient. Orientation or positional relationships indicated by the terms “upper”, “lower”, “top”, “bottom”, “inner”, “outer”, and so on are based on orientation or positional relationships as shown in the drawings, merely for facilitating describing the present disclosure and simplifying the description, rather than indicating or implying that related devices or elements have to be in the specific orientation or configured and operated in a specific orientation, and thus they should not be construed as limitation to the present disclosure. In the description of the present disclosure, “a plurality of” means two or more, and at least one may refer to one, two or more, unless otherwise clearly and specifically specified.

The following disclosure provides many different embodiments or examples for implementing different structures of the present disclosure. In order to simplify the present disclosure, parts and configurations of particular examples are described below. Certainly, they are merely exemplary, and are not intended to limit the present disclosure.

Hemostasis with hemostatic clips is categorized as mechanical hemostasis, that is, a tissue surrounding a wound and bleeding blood vessels are clamped and secured through a closing force between clamping pieces 22 of the hemostatic clip, thereby achieving a hemostatic effect. In practical use, the hemostatic clip is delivered into a lesion site through a channel of an endoscope, the hemostatic clip is utilized to ligate the wound, and then the hemostatic clip is released so as to achieve a purpose of sustained hemostasis.

However, when hemostasis is performed on a large wound, due to a large wound area or rigid scar tissue, a high tension of wound tissue makes it difficult for the hemostatic clip to clamp the wound tissue, even if the wound tissue is clamped, it is still prone to delayed dropping off, and thus the wound cannot be effectively clamped or closed, and the hemostatic effect cannot be achieved.

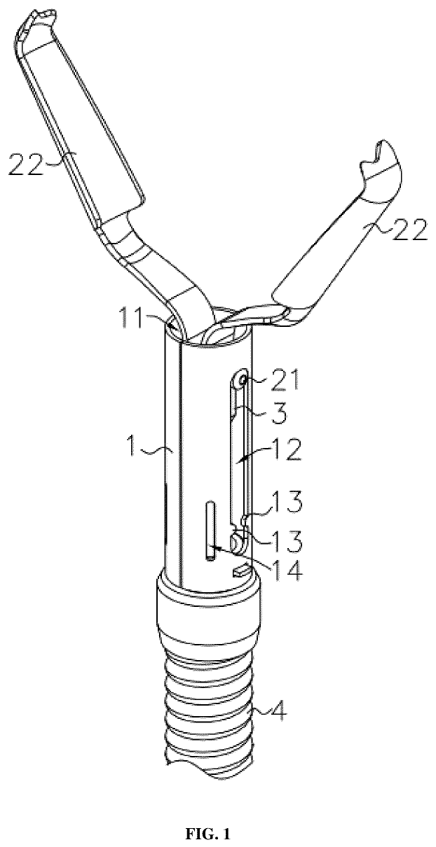

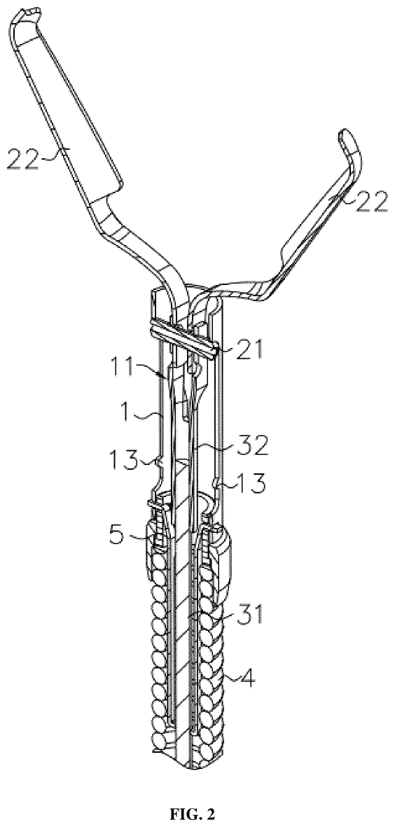

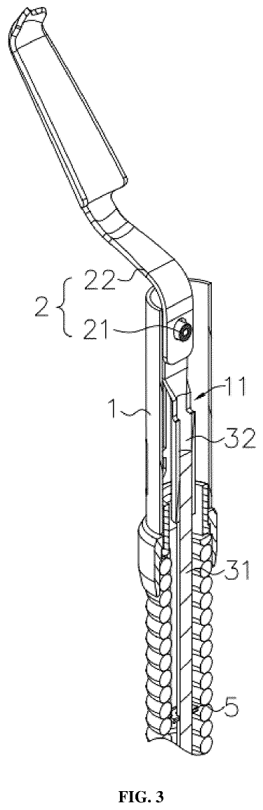

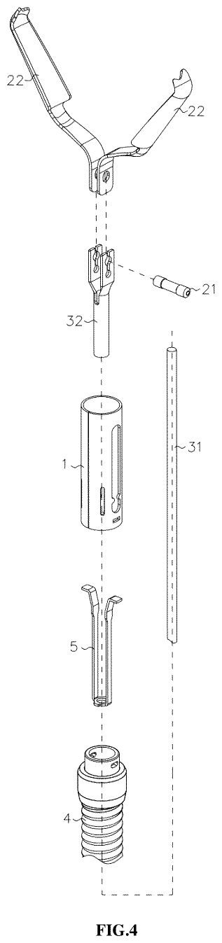

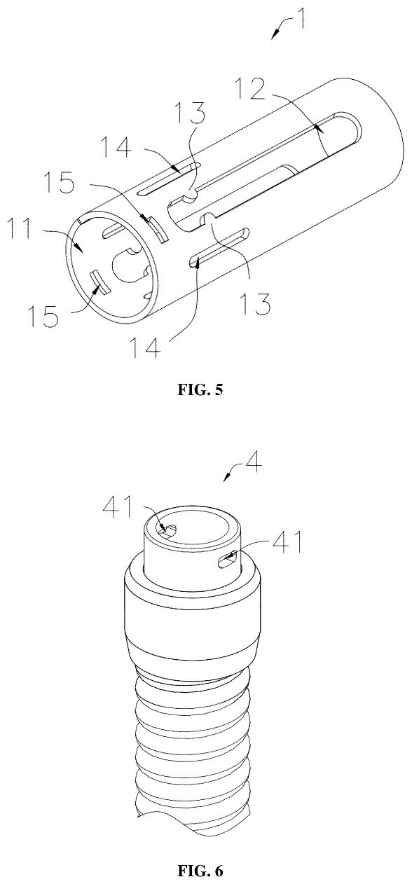

In order to solve the technical problem of loosening and dropping off caused by insecure clamping of the hemostatic clip in hemostasis of a large wound, a first embodiment of the present disclosure provides a hemostatic clip for an endoscope. With reference to FIG. 1, FIG. 2, FIG. 3, FIG. 4 and FIG. 5, FIG. 1 is a structural schematic diagram of a hemostatic clip for an endoscope provided by embodiments of the present disclosure, FIG. 2 is a cross-sectional view of the hemostatic clip for an endoscope provided by embodiments of the present disclosure in one direction, FIG. 3 is a cross-sectional view of the hemostatic clip for an endoscope provided by embodiments of the present disclosure in another direction, FIG. 4 is an exploded schematic view of the hemostatic clip for an endoscope provided by embodiments of the present disclosure, and FIG. 5 is a structural schematic diagram of a clip holder 1 in the hemostatic clip for an endoscope provided by embodiments of the present disclosure. The hemostatic clip for an endoscope includes the clip holder 1 and a clamping assembly 2. The clip holder 1 has a passage 11 provided to extend (e.g., run) therethrough, a guiding slot 12 communicating with the passage 11 and an avoidance slot provided circumferentially along the clip holder 1 and spaced apart from the guiding slot 12. The clip holder 1 includes a first limiting portion 13, where at least part of the first limiting portion 13 is located in the guiding slot 12, and at least part of the first limiting portion 13 is located between the guiding slot 12 and the avoidance slot 14. At least part of the clamping assembly 2 is located in the passage 11, and the clamping assembly 2 includes a movable pin 21 and at least two clips 22 connected to each other, with the movable pin 21 extending through the guiding slot 12. The clamping assembly 2 can move relative to the clip holder 1 along the passage 11, thereby enabling the movable pin 21 to move along the guiding slot 12. In the above, the movable pin 21 can move along the guiding slot 12 to a locking position, the first limiting portion 13 is configured to deform towards the interior of the avoidance slot 14 when subjected to compression of the movable pin 21, and the first limiting portion 13 is configured to limit the movable pin 21 to the locking position, thereby enabling the at least two clips 22 to be closed to each other and locked relative to a position of the clip holder 1.

Specifically, “locking” in the above means that the movable pin 21 is limited to the locking position, so that positions of the two clips 22 can move only within a certain range. Although the movable pin 21 can move to a small extent within the certain range, the two clips 22 can always be kept closed and the clips 22 abut against each other, thereby enabling the hemostatic clip to have a clamping force.

The first limiting portion 13 includes a part located in the guiding slot 12, and the part located in the guiding slot 12 is configured to directly limit the movable pin 21. Specifically, the part of the first limiting portion 13 located in the guiding slot 12 has an arc-shaped surface capable of contacting the movable pin 21, so that the movable pin 21, when subjected to a force in a first direction X towards the proximal end, compresses the clip holder 1, to induce deformation of at least part of the clip holder 1 towards the avoidance slot 14. Consequently, a width of the guiding slot 12 at a position where the first limiting portion 13 is located is increased, thereby enabling the movable pin 21 to pass through the first limiting portion 13 and reach the locking position.

The first limiting portion 13 further includes a part located in the guiding slot 12 and a part located between the guiding slot 12 and the avoidance slot 14. By providing the avoidance slot 14, the part of the first limiting portion 13 located in the guiding slot 12 and the avoidance slot 14 becomes a mechanical weak point, thereby enabling a deformation range of the clip holder 1 under compression to be controllable. Under the compression of the movable pin 21, the deformed part is limited in the first limiting portion 13, that is, the first limiting portion 13 is deformed towards the avoidance slot 14, thus avoiding expansion of the deformation range to cause failure of other parts, thereby improving reliability of the hemostatic clip for an endoscope.

The movable pin 21 in the clamping assembly 2 is always located in the passage 11, while the clips 22 are partially located in the passage 11, and when the more the proximal ends of the clips 22 are located in the passage 11, the closer the end portions of distal ends of the two clips 22 are.

The guiding slot 12 is configured to limit a movement direction of the movable pin 21, such that the movable pin 21 can only move back and forth between a proximal end and a distal end of the clip holder 1.

In some other embodiments, the avoidance slot 14 can also be present in the form of a hole. In the above embodiments, when the movable pin 21 is not located in the locking position, a closing action of the clips 22 and generation of the clamping force need to be realized by an action of movement of the movable pin 21 from the distal end to the proximal end, and when the movable pin 21 is closer to a proximal end of the guiding slot 12, a larger clamping force is generated between the two clips 22. When the movable pin 21 enters the locking position, the clamping force generated between the two clips 22 is the largest, and as the first limiting portion 13 can lock the movable pin 21 having entered the locking position, when the clips 22 and the clip holder 1 are released, the two clips 22 can still be closed and a relatively large clamping force can be maintained between the two clips 22, thus avoiding delayed dropping off during hemostasis of large wounds with the hemostatic clip.

In some embodiments, the first limiting portion 13 is configured to generate elastic deformation when subjected to compression of the movable pin 21, thereby enabling the movable pin 21 to move along the guiding slot 12 and detach from the first limiting portion 13.

Specifically, in order to lock the clips 22, the movable pin 21 is enabled to move from the distal end to the proximal end in the guiding slot 12, and after the movable pin 21 is in contact with the first limiting portion 13, a tensile force to the movable pin 21 is increased, so that the movable pin 21 compresses the first limiting portion 13, the first limiting portion 13 generates elastic deformation, and the movable pin 21 is moved past the first limiting portion 13 into the locking position.

In the above embodiments, after the movable pin 21 is subjected to a force, the deformed part of the clip holder 1 is limited to the first limiting portion 13 so as to reduce the deformation range of the clip holder 1 as much as possible, thereby avoiding failure of other parts due to a large deformation range of the clip holder 1, and further improving the reliability of the hemostatic clip for an endoscope.

In some embodiments, the avoidance slot 14 is provided only on one side of the guiding slot 12, and the guiding slot 12 is correspondingly provided with one first limiting portion 13. A function of limiting the movable pin 21 can be achieved by providing one first limiting portion 13. In this embodiment, when the movable pin 21 passes through the first limiting portion 13, the first limiting portion 13 is deformed, and the width of the guiding slot 12 at the position where the first limiting portion 13 is located is increased, thereby enabling the movable pin 21 to pass therethrough.

In some other embodiments, the clip holder 1 includes a plurality of first limiting portions 13, at least part of the guiding slot 12 is provided between two first limiting portions 13 opposite to each other; and the avoidance slot 14 is provided on one side of the guiding slot 12, thereby enabling the first limiting portion 13 on one side of the guiding slot 12 to correspond to the avoidance slot 14. Thus, the first limiting portion 13 corresponding to the avoidance slot 14 is less deformed, providing better reliability.

In some further embodiments, with reference to FIG. 5 again, the clip holder 1 includes a plurality of first limiting portions 13, at least part of the guiding slot 12 is provided between two first limiting portions 13 opposite to each other; and the avoidance slots 14 are provided on two sides of the guiding slot 12, and each of the first limiting portions 13 is corresponding to one avoidance slot 14.

In the above embodiment, by providing the avoidance slots 14 respectively on two sides of the guiding slot 12, with the same guiding slot 12 corresponding to at least two first limiting portions 13, the elastic deformation generated by the first limiting portions 13 is reduced when the movable pin 21 passes therethrough. Compared with an embodiment in which one guiding slot 12 is corresponding to one first limiting portion 13, in the embodiment in which one guiding slot 12 is corresponding to two first limiting portions 13, the deformation of the first limiting portions 13 is smaller, the elastic deformation is not liable to convert into plastic deformation, and the clip holder 1 and other parts are not prone to failure, thus rendering better reliability of the hemostatic clip for an endoscope.

In some embodiments, referring again to FIG. 1, FIG. 2, FIG. 3 and FIG. 5 again, the locking position is located at the proximal end of the guiding slot 12. When the movable pin 21 is located at a distal end of the guiding slot 12, a plurality of clips 22 are opened relative to each other, and when the movable pin 21 is located at the proximal end of the guiding slot 12, the plurality of clips 22 are closed relative to each other. As can be seen from the preceding, the closer the movable pin 21 is to the proximal end of the guiding slot 12, the larger the clamping force generated between the two clips 22 is. Therefore, providing the locking position at the proximal end of the guiding slot 12 enables the largest clamping force generated between the two clips 22 when the movable pin 21 is located in the locking position, and when the clip holder 1 and the clips 22 are released, a relatively large clamping force can still be maintained between the two clips 22, thus avoiding the occurrence of delayed dropping off.

In some embodiments, referring to FIG. 4 again, the hemostatic clip for an endoscope further includes a control assembly 3, where at least part of the control assembly 3 is located in the passage 11, and the movable pin 21 is connected to the control assembly 3; and the control assembly 3 can move relative to the clip holder 1 along the passage 11, so as to drive the clamping assembly 2 to move.

In the above embodiments, the control assembly 3 capable of moving along the passage 11 is configured to move the movable pin 21 so as to close and open the two clips 22, that is, the movement of the movable pin 21 can be controlled by controlling the movement of the control assembly 3, thereby controlling the opening and closing between the two clips 22 and controlling a magnitude of the clamping force. When the control assembly 3 is moved from the proximal end to the distal end, the clamping force between the two clips 22 changes from strong to weak, causing the two clips 22 to open from closing; when the control assembly 3 is moved from the distal end to the proximal end, the clamping force between the two clips 22 changes from weak to strong, causing the two clips 22 to close from opening.

In some embodiments, referring to FIG. 8, FIG. 8 is a schematic structural diagram of the movable pin 21 in the hemostatic clip for an endoscope provided by embodiments of the present disclosure. The movable pin 21 includes a connecting portion 211 and two second limiting portions 212, and two ends of the connecting portion 211 are connected to the two second limiting portions 212, respectively. The clips 22 and the control assembly 3 are all connected to the connecting portion 211, and the second limiting portions 212 are configured to limit the clips 22 and the control assembly 3 on the connecting portion 211 in an extending direction X of the movable pin 21, where the extending direction X of the movable pin 21 is a direction of an X axis in FIG. 8.

Specifically, each of the two second limiting portions 212 has a limiting surface, and two limiting surfaces are provided opposite to each other.

Further, each of the second limiting portions 212 has a cylindrical surface, an outside diameter of the second limiting portions 212 is larger than an outside diameter of the connecting portion 211, and the second limiting portions 212 and the connecting portion 211 are provided coaxially and connected axially.

In the above embodiments, the limiting surfaces can be connected to the control assembly 3 so as to limit the clips 22 and the control assembly 3 to the connecting portion 211. Connection between the control assembly 3 and the limiting surfaces can also axially limit the movable pin 21, thus avoiding the movable pin 21 from moving axially to be separated from the clips 22 or the control assembly 3. In addition, the cylindrical surfaces of the second limiting portions 212, when compressing the first limiting portion 13, can play a guiding role, thereby enabling the second limiting portions 212 to pass through the first limiting portion 13 more smoothly.

In some embodiments, the control assembly 3 includes a pulling wire 31 and a clevis 32, the movable pin 21 is connected to the clevis 32, the clevis 32 has a first fitting hole 321, and the pulling wire 31 is embedded in the first fitting hole 321 so as to be connected to the clevis 32. When the movable pin 21 is located in the locking position and the pulling wire 31 is subjected to a preset tensile force, the pulling wire 31 can be detached from the first fitting hole 321, thereby disconnecting the pulling wire 31 from the clevis 32.

Specifically, the clevis 32 is configured to drive the movable pin 21 to move, the pulling wire 31 is configured to transmit power, and the clevis 32 is a flexible connector capable of bearing certain deformation, so that the pulling wire 31 can also be bent when a catheter 4 is bent. The pulling wire 31 and the clevis 32 are limited through frictional force, the preset tensile force is greater than static frictional force between the pulling wire 31 and the clevis 32, and the preset tensile force is greater than the tensile force required for the movable pin 21 to pass through the first limiting portion 13. It can be understood that when the pulling wire 31 is subjected to a relatively small tensile force, the clevis 32 can draw the movable pin 21 to move in the guiding slot 12. When the movable pin 21 is in contact with the first limiting portion 13, the tensile force applied to the pulling wire 31 needs to be increased, so as to enable the movable pin 21 to compress the first limiting portion 13, and consequently, the first limiting portion 13 is deformed, and the movable pin 21 passes through the first limiting portion 13 to enter the locking position. In this case, as the pulling wire 31 is subjected to a tensile force smaller than the preset tensile force, the pulling wire 31 is not separated from the clevis 32. When the clips 22 need to be released, a tensile force is applied to the pulling wire 31, and the clevis 32 is pulled by the pulling wire 31 to come into contact with a locking member 5, thus increasing the tensile force on the pulling wire 31, enabling the clevis 32 to push the locking member 5 towards the proximal end, such that the catheter 4 is separated from the clip holders 1. When the catheter 4 is separated from the clip holder 1, or after the catheter 4 is separated from the clip holder 1, the preset tensile force greater than the static frictional force between the pulling wire 31 and the clevis 32 is applied to the pulling wire 31, a relative movement is generated between the pulling wire 31 and the clevis 32, and the pulling wire 31 is separated from the clevis 32, thus completing the release of the clips 22 and the clip holder 1.

In the above embodiments, the pulling wire 31 and the clevis 32 are limited relative to each other through the first fitting hole 321 and the frictional force, so that it is easy to separate the pulling wire 31 and the clevis 32. The pulling wire 31 and the clevis 32 can be separated only by applying a tensile force greater than the frictional force, thus simplifying structures of the pulling wire 31 and the clevis 32.

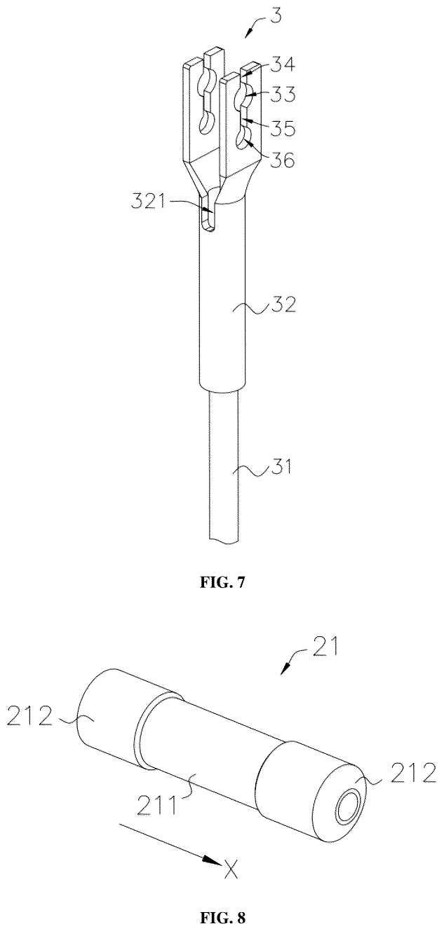

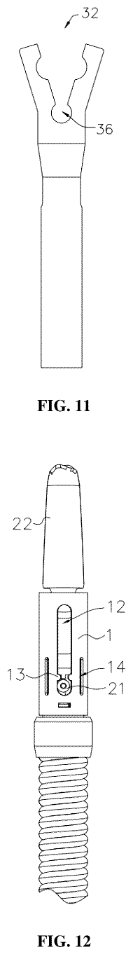

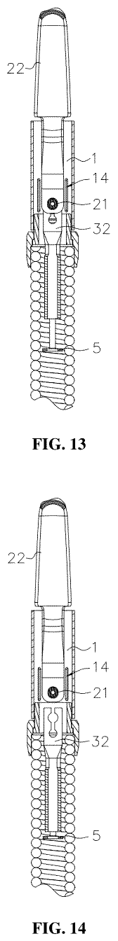

In some other embodiments, referring to FIG. 7, FIG. 12, FIG. 13 and FIG. 14, FIG. 7 is a structural schematic diagram of the control assembly 3 in the hemostatic clip for an endoscope provided by embodiments of the present disclosure, FIG. 12 is a structural schematic diagram of the hemostatic clip for an endoscope provided by embodiments of the present disclosure, with the movable pin being located in the locking position, FIG. 13 is a cross-sectional view of the hemostatic clip for an endoscope provided by embodiments of the present disclosure, with the movable pin being located in the locking position, and FIG. 14 is a cross-sectional view of the hemostatic clip for an endoscope provided by embodiments of the present disclosure, with the movable pin being separated from the control assembly. The control assembly 3 has a second fitting hole 33 and a through slot 34 communicating with the second fitting hole 33, the through slot 34 extends from the fitting hole towards a distal end of the control assembly 3, and the movable pin 21 extends through the second fitting hole 33. When the movable pin 21 is located in the locking position and the control assembly 3 is subjected to the preset tensile force, the control assembly 3 can move along the through slot 34 relative to the movable pin 21, thereby enabling the movable pin 21 to be separated from the control assembly 3 via the through slot 34.

Specifically, the second fitting hole 33 and the through slot 34 are both provided at a distal end of the clevis 32, the distal end of the clevis 32 has two side walls opposite to each other, the second fitting hole 33 and the through slot 34 are both provided at side walls, a connection space is formed between the two side walls, and the proximal ends of the clips 22 are provided in the connection space.

Specifically, when the control assembly 3 is subjected to the preset tensile force, as the movable pin 21 already has been located in the locking position, blocked by the clip holder 1, the movable pin 21 cannot move further towards the proximal end, and the control assembly 3 has a tendency to move towards the proximal end under the action of the preset tensile force, and the movable pin 21 compresses parts of the control assembly 3 located at two sides of the through slot 34, so that the parts of the control assembly 3 located at the two sides of the through slot 34 are deformed, or the movable pin 21 is deformed, or the movable pin 21 and the control assembly 3 are deformed concurrently, and the movable pin 21 is detached from the control assembly 3 from the through slot 34, thus realizing separation of the control assembly 3 from the movable pin 21.

Further, deformation generated by the parts of the control assembly 3 located at the two sides of the through slot 34 may be elastic deformation or plastic deformation. In order to ensure that the movable pin 21 can remain in the locking position after being separated from the control assembly 3, the deformation generated by the movable pin 21 is elastic deformation, so as to allow the movable pin 21 to regain its original shape after being separated from the control assembly 3, and to be limited at a proximal end of the first limiting portion 13.

Specifically, the connecting portion 211 of the movable pin 21 has a cylindrical surface, and the cylindrical surface can play a guiding role when the movable pin 21 passes through the through slot 34.

In the above embodiments, by providing the through slot 34 on the control assembly 3, a passage 11 is provided for the movable pin 21 to move away from the second fitting hole 33 when the clips 22 need to be released. Furthermore, when the clips 22 do not need to be released, the control assembly 3 still has a relatively reliable limiting effect on the movable pin 21, thereby enabling the hemostatic clip for an endoscope to have relatively good reliability.

In some embodiments, referring to FIG. 10 and FIG. 11, FIG. 10 is a structural schematic diagram of the clevis 32 in the hemostatic clip for an endoscope provided by embodiments of the present disclosure, and FIG. 11 is a structural schematic diagram of the hemostatic clip for an endoscope provided by embodiments of the present disclosure, after deformation of the clevis. A width of the movable pin 21 is greater than that of the through slot 34, and when the movable pin 21 is located in the through slot 34, the movable pin 21 compresses the control assembly 3 to cause the control assembly 3 to generate deformation, thereby enabling the movable pin 21 to be detached from the through slot 34 and to be separated from the control assembly 3.

Further, deformation of the parts of the control assembly 3 located at the two sides of the through slot 34 may be elastic deformation or plastic deformation.

It can be understood that the control assembly 3 has a small thickness at a position connected to the movable pin 21, and compared with the cylindrical movable pin 21, the part of the control assembly 3 connected to the movable pin 21 is a mechanical weak part, which is more prone to deformation.

In the above embodiments, it is defined that when the movable pin 21 is detached from the control assembly 3, the control assembly 3 at least generates deformation, which can reduce the preset tensile force required for separating the control assembly 3 from the movable pin 21.

In some embodiments, referring to FIG. 7, FIG. 10 and FIG. 11 again, the control assembly 3 has an elongate slot 35 communicating with a side of the second fitting hole 33 away from the through slot 34, and the elongate slot 35 extends from the fitting hole towards a proximal end of the control assembly 3.

Specifically, the elongate slot 35 is provided on the clevis 32.

In the above embodiments, the control assembly 3 is provided with the elongate slot 35 communicating with the second fitting hole 33, and the elongate slot 35 communicates with a distal end of the second fitting hole 33, such that a range of the mechanical weak portion is enlarged, a width of the through slot 304 after deformation of the control assembly 3 can be wider, and a deformation range of the control assembly 3 subjected to the same preset tensile force is larger. After the control assembly 3 is subjected to the same preset tensile force, the control assembly 3 provided with the elongate slot 35 can be more easily separated from the movable pin 21, that is, operation difficulty of the hemostatic clip for an endoscope is reduced.

In some embodiments, referring to FIG. 7, FIG. 10 and FIG. 11 again, the control assembly 3 has a stopper hole 36 communicating with the elongate slot 35, and the stopper hole 36 communicates with a side of the elongate slot 35 away from the second fitting hole 33. Specifically, the stopper hole 36 is provided on the clevis 32.

It can be understood that a proximal end of the elongate slot 35 is likely to generate a concentrated stress to cause failure of the control assembly 3, providing the stopper hole 36 communicating with the proximal end of the elongate slot 35 can avoid the failure of the control assembly 3, thereby improving the reliability of the hemostatic clip for an endoscope.

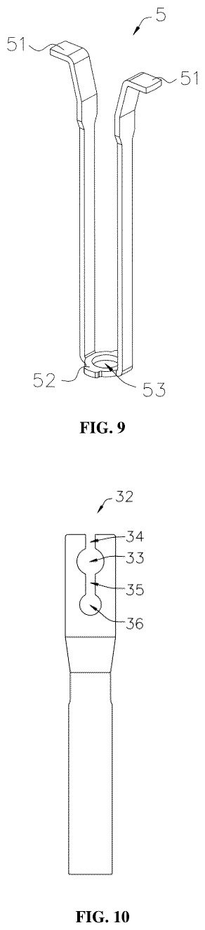

In some embodiments, referring to FIG. 4 and FIG. 9 again, FIG. 9 is a structural schematic diagram of the locking member 5 in the hemostatic clip for an endoscope provided by embodiments of the present disclosure. The hemostatic clip for an endoscope includes the catheter 4 and the locking member 5, the control assembly 3 extends through a lumen of the catheter 4, the catheter 4 has a first connection hole 41 (shown in FIG. 6), the clip holder 1 has a second connection hole 15, and the locking member 5 extends through the first connection hole 41 and the second connection hole 15 so as to connect the catheter 4 and the clip holder 1.

Referring to FIG. 14 again, when the movable pin 21 is located in the locking position and the control assembly 3 is subjected to the preset tensile force, the control assembly 3 can abut against and push the locking member 5 after being detached from the movable pin 21, thereby enabling the locking member 5 to be detached from the first connection hole 41 and the second connection hole 15, and separating the catheter 4 from the clip holder 1.

Specifically, the locking member 5 includes at least two jaws 51 and a base 52. The jaws 51 are configured to extend through the first connection hole 41 and the second connection hole 15, the base 52 is provided with a through hole 53 for the pulling wire 31 to penetrate therethrough, and the base 52 is configured to abut against the clevis 32 which has been separated from the movable pin 21. Herein, each of the jaws 51 has a guiding slope, so that when the base 52 is compressed by the clevis 32, the jaws 51 can move towards the pulling wire 31 to detach the jaws 51 from at least one of the first connection hole 41 and the second connection hole 15, thus separating the clip holder 1 from the catheter 4.

In the above embodiments, by providing the locking member 5, the clip holder 1 can be separated from the catheter 4, thus releasing the clips 22, and leaving the clips 22 at the wound for sustained hemostasis.

A usage procedure of the hemostatic clip for an endoscope is as follows:
1. controlling the movable pin 21 to be located at one side of the first limiting portion 13 close to the distal end of the clip holder 1, so as to avoid the movable pin 21 from entering the locking position, and also to keep the two clips 22 to be closed;
2. putting the hemostatic clip for an endoscope into the channel so as to deliver the hemostatic clip into human body through the channel;
3. when a distal end of the channel reaches the vicinity of the wound, stretching out the hemostatic clip for an endoscope;
4. pushing the movable pin 21 to the distal end of the guiding slot 12, so as to open the two clips 22 and get ready to clamp the wound;
5. controlling the movable pin 21 to move from the distal end of the guiding slot 12 to the proximal end, thereby enabling the two clips 22 to close and clamp the wound;
6. controlling the movable pin 21 to penetrate through the first limiting portion 13 to move to the locking position, with reference to FIG. 12 and FIG. 13;
7. applying the preset tensile force to the pulling wire 31, thereby enabling the clevis 32 to be separated from the movable pin 21, with reference to FIG. 14; and
8. continuing to apply the tensile force to the pulling wire 31, thereby enabling the clevis 32 to abut against the locking member 5, separating the clip holder 1 from the catheter 4, and completing the release of the clips 22 and the clip holder 1, so as to leave the clips 22 on the wound, and continuously apply a clamping force to the wound for hemostasis of the wound.

The hemostatic clip for an endoscope provided by the embodiments of the present disclosure is introduced in detail in the above. Specific examples are utilized in the present disclosure to illustrate the principle and embodiments of the present disclosure. The description of the above embodiments is only intended to help understanding the technical solution of the present disclosure and the core idea thereof. Those ordinarily skilled in the art should understand that they still could modify the technical solutions described in various preceding embodiments, or make equivalent substitutions to some or all of the technical features therein; and these modifications or substitutions do not make the essence of corresponding technical solutions depart from the scope of the technical solutions of various embodiments of the present disclosure.

1. A hemostatic clip for an endoscope, comprising:
a clip holder, wherein the clip holder comprises a passage that runs therethrough, a guiding slot communicating with the passage, and an avoidance slot provided circumferentially along the clip holder and spaced apart from the guiding slot, wherein the clip holder comprises a first limiting portion, at least part of the first limiting portion is located in the guiding slot, and at least part of the first limiting portion is located between the guiding slot and the avoidance slot; and
a clamping assembly, wherein at least part of the clamping assembly is located in the passage, and the clamping assembly comprises a movable pin and at least two clips, wherein the movable pin and the at least two clips are connected to each other, and the movable pin extends through the guiding slot; the clamping assembly is movable relative to the clip holder along the passage, so as to make the movable pin movable along the guiding slot,
wherein the movable pin is movable along the guiding slot to a locking position, the first limiting portion is configured to deform towards an interior of the avoidance slot when subjected to compression of the movable pin, and the first limiting portion is configured to limit the movable pin to the locking position, so that the at least two clips are closed to each other and locked relative to a position of the clip holder.

2. The hemostatic clip for an endoscope according to claim 1, wherein the first limiting portion is configured to generate elastic deformation when subjected to compression of the movable pin, so as to make the movable pin movable along the guiding slot and detach from the first limiting portion.

3. The hemostatic clip for an endoscope according to claim 1, wherein the clip holder comprises a plurality of first limiting portions, at least part of the guiding slot is provided between two first limiting portions opposite to each other.

4. The hemostatic clip for an endoscope according to claim 1, wherein the locking position is located at a proximal end of the guiding slot.

5. The hemostatic clip for an endoscope according to claim 1, wherein the hemostatic clip for an endoscope further comprises:
a control assembly, wherein at least part of the control assembly is located in the passage, and the movable pin is connected to the control assembly; and the control assembly is movable relative to the clip holder along the passage, so as to drive the clamping assembly to move.

6. The hemostatic clip for an endoscope according to claim 5, wherein the movable pin comprises a connecting portion and two second limiting portions, and two ends of the connecting portion are connected to the two second limiting portions, respectively; and
the clips and the control assembly are all connected to the connecting portion, and the second limiting portions are configured to limit the clips and the control assembly on the connecting portion in an extending direction of the movable pin.

7. The hemostatic clip for an endoscope according to claim 5, wherein the control assembly comprises a pulling wire and a clevis, the movable pin is connected to the clevis, the clevis is formed with a first fitting hole, and the pulling wire is embedded in the first fitting hole so as to be connected to the clevis; and
when the movable pin is located in the locking position and the pulling wire is subjected to a preset tensile force, the pulling wire is capable of being detached from the first fitting hole, so as to disconnect the pulling wire from the clevis.

8. The hemostatic clip for an endoscope according to claim 5, wherein the control assembly is formed with a second fitting hole and a through slot communicating with the second fitting hole, the through slot extends from the fitting hole towards a distal end of the control assembly, and the movable pin extends through the second fitting hole; and
when the movable pin is located in the locking position and the control assembly is subjected to a preset tensile force, the control assembly is movable relative to the movable pin along the through slot, so as to make the movable pin separate from the control assembly via the through slot.

9. The hemostatic clip for an endoscope according to claim 8, wherein a width of the movable pin is greater than that of the through slot, and when the movable pin is located in the through slot, the movable pin compresses the control assembly to cause the control assembly to generate deformation, thereby enabling the movable pin to be detached from the through slot and separated from the control assembly.

10. The hemostatic clip for an endoscope according to claim 9, wherein the control assembly is formed with an elongate slot communicating with a side of the second fitting hole away from the through slot, and the elongate slot extends from the fitting hole towards a proximal end of the control assembly.

11. The hemostatic clip for an endoscope according to claim 10, wherein the control assembly is formed with a stopper hole communicating with the elongate slot, and the stopper hole communicates with a side of the elongate slot away from the second fitting hole.

12. The hemostatic clip for an endoscope according to claim 5, wherein the hemostatic clip for an endoscope comprises a catheter and a locking member, the control assembly extends through a lumen of the catheter, the catheter is formed with a first connection hole, the clip holder has a second connection hole, and the locking member extends through the first connection hole and the second connection hole so as to connect the catheter and the clip holder; and
when the movable pin is located in the locking position and the control assembly is subjected to a preset tensile force, the control assembly is capable of abutting against and pushing the locking member, so as to make the locking member detach from the first connection hole and the second connection hole, and make the catheter separate from the clip holder.
